(19) United States
(12) Patent Application Publication
(10) Pub. No.: US 2003/0087430 A1
(43) Pub. Date: May 8, 2003

(54) Mammalian sterol synthesis as a target for chemotherapy against bacteria

(76) Inventors: Drew Catron, Chicago, IL (US); Kasturi Haldar, Chicago, IL (US); Yvonne Lange, Chicago, IL (US)

Correspondence Address: KLAUBER & JACKSON, 411 HACKENSACK AVENUE, HACKENSACK, NJ, 07601

(21) Appl. No.: 10/268,060

(22) Filed: Oct. 9, 2002

Related U.S. Application Data
(60) Provisional application No. 60/328,765, filed on Oct. 12, 2001.

(51) Int. Cl. C12N 5/06

(52) U.S. Cl. 435/340

(57) ABSTRACT

The present invention discloses methods for treating, ameliorating, or preventing having an infection due to an intracellular vacuolar bacterium. The invention further exemplifies the use of mevinolin (lovastatin) in the treatment of intracellular vacuolar bacterial infections.

### STATEMENT OF GOVERNMENT SUPPORT

The research leading to the present invention was supported, at least in part, by a grant from the National Institutes of Health, Grant Nos. 0600-300-D583 and GM 08061-18. Accordingly, the Government may have certain rights in the invention.

### FIELD OF THE INVENTION

The present invention relates to the treatment of bacterial infections due to intracellular bacteria, and to treating patients having a bacterial infection by inhibiting host cell sterol biosynthesis.

### BACKGROUND OF THE INVENTION

Bacterial infections remain among the most common and deadly causes of human disease. Infectious diseases are the third leading cause of death in the United States and the leading cause of death worldwide (Binder et al. (1999) Science 284:1311-1313). Although, there was initial optimism in the middle of the last century that diseases caused by bacteria would be quickly eradicated, it has become evident that the so-called “miracle drugs” are not sufficient to accomplish this task. Antibiotic resistant pathogenic strains of bacteria have become commonplace, and bacterial resistance to the new variations of these drugs appears to be outpacing the ability of scientists to develop effective chemical analogs of the existing drugs (see, for example, Levy (March 1998) Scientific American, pp. 46-53). Therefore, new approaches to drug development are necessary to combat the ever-increasing number of antibiotic-resistant pathogens.

Salmonella enterica is a significant cause of morbidity and mortality in the United States, responsible for 800,000 to 4 million cases each year. Serovar typhimurium accounts for over 25% of these cases (see, for example, National Antimicrobial Resistance Monitoring System, 1999 Annual Report). Whereas most Salmonella infections are self-limiting, acute intestinal inflammations, serious bacteremia can result in 3% to 10% of the infections. Dissemination most often occurs in young children, the elderly or those who are immunocompromised. In these cases, fluoroquinolones (i.e., ciprofloxacin) and cephalosporins (i.e., ceftriaxone) are commonly used for treatment. However, the rise of drug-resistant S. typhimurium strains is cause for great concern. More specifically, there is an increased incidence of a distinct multidrug-resistant form of typhimurim (DT104), which is resistant to five different antibiotics (ampicillin, chloramphenicol, streptomycin, sulfonamides and tetracyline) (Glynn et al. (1998) N. Engl. J. Med.).

### BRIEF SUMMARY OF THE INVENTION

In the last several years, this strain has also been shown to have significant resistance to ciprofloxacin and ceftriaxone. With widespread drug resistance, derivative anti-bacterials only provide short-term solutions. Therefore, there is an urgent need to identify and target components that bacteria require for intracellular survival. More particularly, there is a need to provide compounds for treating drug-resistant typhimurium strains. Furthermore, there is a need to provide methods for administrating such compounds.

The present invention provides methods of treating, ameliorating, and/or preventing bacterial infections that are at least partially due to intracellular vacuolar bacteria. The present invention further provides methods of treating animal subjects that have a bacterial infection by inhibiting host sterol biosynthesis. Accordingly, in one aspect, the invention provides a method of treating an intracellular vacuolar bacterial infection, comprising administering to a subject in need thereof, a pharmaceutical composition comprising an agent that inhibits one or more steps of the sterol biosynthetic pathway between the biosynthesis of HMG-CoA from acetoacetyl-CoA and acetyl-CoA, and the conversion of squalene to squalene 2,3-oxide (see FIG. 1). In one embodiment, the reaction step(s) inhibited is in the conversion of HMG-CoA from acetoacetyl-CoA and acetylCoA. In another embodiment, the reaction step(s) inhibited is in the conversion of mevalonate to farnesyl pyrophosphate. In yet another embodiment, the reaction step(s) inhibited is in the conversion of farnesyl pyrophosphate to squalene. In still another embodiment the reaction step(s) inhibited is the conversion of squalene to squalene 2,3-oxide. In a preferred embodiment the reaction step(s) inhibited is in the conversion of HMG-CoA to mevalonate. In a more preferred embodiment, the agent is an inhibitor of HMG-CoA reductase.

In one embodiment, the agent is an antibody raised against an enzyme in the sterol biosynthetic pathway between the biosynthesis of HMG-CoA from acetoacetyl-CoA and acety-CoA, and the conversion of squalene to squalene 2,3-oxide. The antibody may be a monoclonal, polyclonal, chimeric and/or humanized antibody.

In one embodiment, the agent is a small organic compound. In a more specific embodiment, the small organic compound is a statin. More specifically, the statin may be selected from the group consisting of cerivastatin, fluvastatin, atorvastatin, simvastatin, pravastatin, and mevinolin (lovastatin).

In one embodiment, a therapeutically effective dose of a statin is effective in inhibiting the growth of an intracellular vacuolar bacteria. As shown in the experiments below, a therapeutically effective does of a statin for treating an intracellular vacuolar infection is lower than the use of statins for other indications, for example less than 50 μM; more preferably, less than 100 nM; even more preferably, 50 nM.

In one embodiment of the method of the invention, a statin is coordinately administered to the animal subject with L-camitine or an alkanoyl L-camitine, in which the linear or branched alkanoyl has 2-6 carbon atoms, or one of their pharmaceutically acceptable salts.

In one embodiment of the method of the invention, the bacterial infections is a Salmonella enterica infection. In a more specific embodiment, the Salmonella enterica infection is due to a Serovar typhimurium. In another embodiment, the bacterial infection is due to Legionella pneumophilum (the causative agent in Legionaire's disease), a Mycobacterium, Coxiellum, Chlamydium, and/or Campylobacter. In one embodiment, the intracellular vacuolar bacterium is an anaerobe. In a preferred embodiment, the subject treated is a human.

Other objects and advantages will become apparent from a review of the ensuing detailed description taken in conjunction with the following illustrative drawing.

### BRIEF DESCRIPTION OF THE DRAWINGS

FIG. 1 is a schematic depiction of the mammalian sterol biosynthetic pathway. The steps between acetyl CoA and cholesterol of the sterol biosynthetic pathway are shown, although many intermediates are omitted for the sake of simplicity.

FIG. 2 demonstrates that mevinolin (lovastatin) inhibits intracellular growth of Salmonella in macrophages. RAW 264.7 macrophages incubated for 6 hours prior to infection with various treatments (DMEM-FBS, DMEM-LPDS, DMEM-D-FBS+30 uM Mevinolin, or DMEM-FBS+30 uM Mevinolin, or DMEM-FBS+30 uM Mevinolin+150 uM mevalonate).

FIG. 3 shows that 4,4,10-β trimethyl-trans-decal-3β-ol (TMD) does not inhibit intracellular growth of Salmonella.

FIGS. 4A-4B show that mevinolin does not inhibit extracellular growth of Salmonella. Optical density measurements of Salmonella grown in plain Luria broth (LB) or LB containing 30 uM Mevinolin (FIG. 4A) or in plain LB (diamonds) or in LB containing 30 uM Mevinolin (triangles) (FIG. 4B).

FIG. 5 shows that mevinolin does not compromise host cell viability. RAW 264.7 macrophages were treated with Mevinolin with or without mevalonate as follows: 1=10 nM mevinolin, 2=100 nM mevinolin, 3=500 nM mevinolin, 4=1 uM mevinolin, 5=10 uM mevinolin, 6=50 uM mevinolin, 7=100 uM mevinolin, 8=50 uM mevinolin+50 uM mevalonate, 9=50 uM mevinolin+150 uM mevalonate, 10=100 uM mevinolin+50 uM mevalonate, and 11=100 uM mevinolin+150 uM mevalonate.

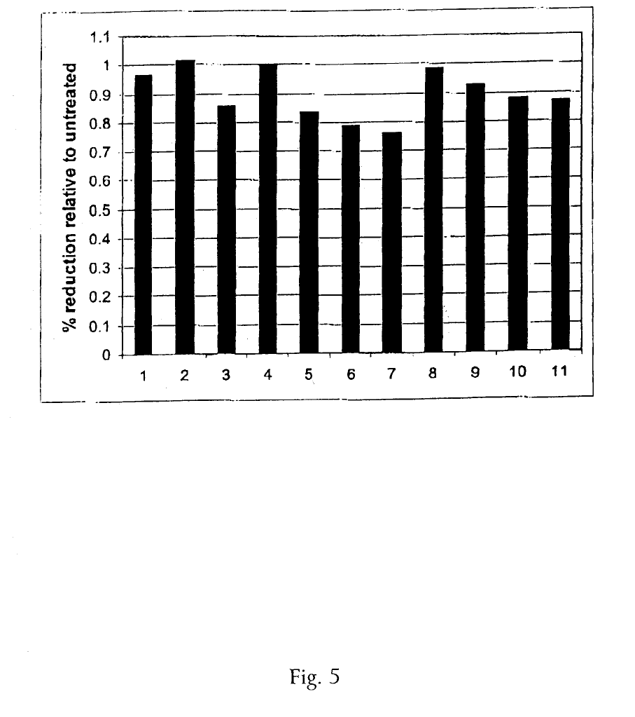

FIG. 6 shows that mevinolin inhibits intracellular growth of Legionella in human macrophages. U937 macrophages were treated with RPMI-FBS, RPMI-FBS+30 uM mevinolin, or RPMI-FBS+30 uM mevinolin+150 uM mevalonate.

FIG. 7 shows mevinolin induces apoptosis in Salmonella-infected macrophages. RAW264.7 macrophages were treated with 30 μM mevinolin, and then infected with S. typhimurium. TUNEL staining scores are shown for uninfected cells and infected cells.

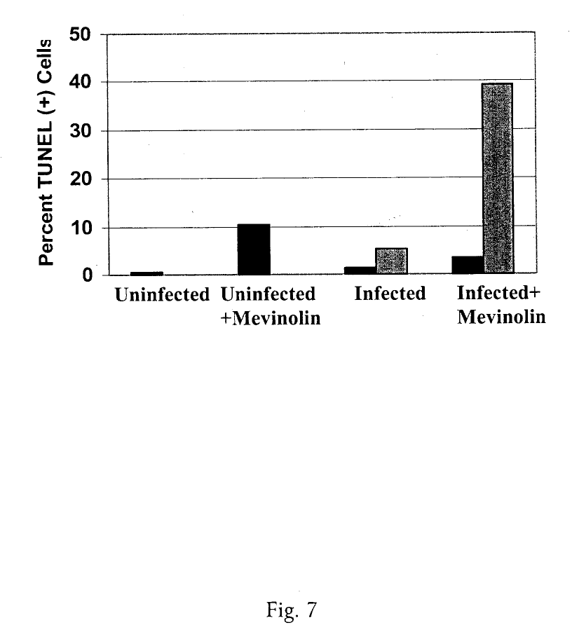

FIG. 8 shows that physiologically relevant concentrations of mevinolin inhibit the intracellular growth of Salmonella. TUNEL staining scores are shown for uninfected and infected cells treated with 50 nM mevinolin.

### DETAILED DESCRIPTION

Before the present method methodology is described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Definitions

As used herein, a “small organic molecule” is an organic compound, or organic compound complexed with an inorganic compound such as a metal, that has a molecular weight of less than 3 kilodaltons, and preferably less than 1.5 kilodaltons. A “compound” of the present invention is preferably a small organic molecule.

As used herein, an “intracellular vacuolar bacterium” is a bacterium that lives in a vacuole of its host cell. Although, intracellular vacuolar bacteria can also live in an extracellular enviroment, an intracellular infection is critical for establishing their systemic infections. Examples of intracellular, vacuolar bacteria include Salmonella, Legionella, Mycobacterium, Coxiella, Chlamydia, and Campylobacter.

As used herein, “statins” are small organic compounds that (i) inhibit the enzyme HMGCoA reductase and (ii) have a common chemical structure, as exemplified by the natural fermentation product lovastatin, and simvastatin, (see U.S. Pat. Nos. 4,231,938, and 5,763,646, the contents of which are herein specifically incorporated by reference in their entirety). Statins can be used as antihypercholesterolenic agents and include such commercially available drugs as lovastatin/mevinolin (MEVACOR™), fluvastatin (LESCOL™), cerivastatin (BAYCOL™), atorvastatin (LIPITOR™), simvastatin (ZOCOR™) and pravastatin (PRAVACHOL™). As used herein “mevinolin” and “lovastatin” are used interchangeably and denote the chemical compound known as mevinolin.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host, i.e., a bacterial infection.

The term “therapeutically effective dose” means a dose that produces the desired effects for which it is administered. The exact dose will depend on the purpose of the treatment, and will be ascertainable by one skilled in the art using known techniques (see, for example, Lieberman (1992) Pharmaceutical Dosage Forms Vol. 1-3; Lloyd (1999) The Art, Science and Technology of Pharmaceutical Compounding; and Pickar (1999) Dosage Calculations). The invention is based, in part, on the discovery that a very low dose of an inhibitor of one or more of the early sterol biosynthetic pathway steps, such as a statin, is effective in treating an infection caused by a the presence of an intracellular vacuolar bacterium. The phrase “physiological dose” or “physiologically relevant” concentrations or doses refer to low concentrations of a statin such as mevinolin in inhibiting intracellular bacterial growth, e.g., a concentration of less than 100 μM, or more preferably, a concentration of 50 μM or less. More specifically, a therapeutically effective dose of a statin for the treatment of an intracellular vacuolar infection is preferably less than 100 nM; even more preferably, 50 nM.

The present invention provides the novel use of agents which inhibit mammalian sterol biosynthesis to treat and/or prevent bacterial diseases caused by intracellular vacuolar bacteria. The present invention further provides a new use for statins, i.e., in the treatment and/or prevention of intracellular vacuolar bacterial infections, since statins are known to inhibit mammalian sterol biosynthesis.

Current anti-bacterial drugs are directed against various targets within the bacterium itself, such as replication or cell wall synthesis. Unfortunately, bacterial resistance to all classes of such antibiotics has emerged. As disclosed herein, however, the survival of intracellular vacuolar bacteria in a host cell, as exemplified by Salmonella below, depends upon one or more steps of the sterol biosynthetic pathway of the host cell. Thus, by targeting the host sterol metabolic pathway required for the survival of an intracellular vacuolar bacterium in its host cell, e.g., Salmonella in macrophages, the present invention provides a way to kill bacteria and/or hinder bacterial growth that is radically different from current treatment methods.

Antibodies

According to the present invention, the proteins/enzymes involved in the sterol biosynthetic pathway between the biosynthesis of HMG-CoA from acetoacetyl-CoA and acetylCoA, and the conversion of squalene to squalene 2,3-oxide may be used as an immunogen to generate antibodies. In a particular embodiment, the antibody is raised against HMG-CoA reductase and inactivates this enzyme when bound thereto. Such antibodies include but are not limited to polyclonal, monoclonal, chimeric including humanized chimeric, single chain, Fab fragments, and a Fab expression library. The antibodies of the invention may be cross reactive, that is, they may recognize the same protein derived from a different source. Polyclonal antibodies have greater likelihood of cross reactivity. Alternatively, an antibody of the invention may be specific for a single form of an enzyme, such as the human HMG-CoA reductase.

Various procedures known in the art may be used for the production of polyclonal antibodies to the proteins/enzymes involved in the sterol biosynthetic pathway between the biosynthesis of HMG-CoA from acetoacetyl-CoA and acetyl-CoA, and the conversion of squalene to squalene 2,3-oxide, or derivatives or analogs of these proteins/enzymes. For the production of antibody, various host animals can be immunized by injection with the protein/enzyme, or a derivative (e.g., or fusion protein) thereof, including but not limited to rabbits, mice, rats, sheep, goats, etc. In one embodiment, the protein/enzyme or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the proteins/enzymes involved in the sterol biosynthetic pathway between the biosynthesis of HMG-CoA from acetoacetyl-CoA and acetyl-CoA, and the conversion of squalene to squalene 2,3-oxide, or analog, or derivative thereof, any technique that provides for the production of antibody molecules by continuous cell lines in culture may be used. These include but are not limited to the hybridoma technique originally developed by Kohler and Milstein (1975) Nature 256:495-497), as well as the trioma technique, the human B-cell hybridoma technique (Kozbor et al. (1983) Immunology Today, 4:72; Cote et al. (1983) Proc. Natl. Acad. Sci. U.S.A. 80:2026-2030), and the EBV-hybridoma technique to produce human monoclonal antibodies (Cole et al. (1985) in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). In fact, according to the invention, techniques developed for the production of “chimeric antibodies” (Morrison et al. (1984) J. Bacteriol. 159:870; Neuberger et al. (1984) Nature 312:604-608; Takeda et al. (1985) Nature 314:452-454) by splicing the genes from a mouse antibody molecule specific for a HMG-CoA reductase, for example, together with genes from a human antibody molecule of appropriate biological activity can be used; such antibodies are within the scope of this invention.

According to the invention, techniques described for the production of single chain antibodies (U.S. Pat. Nos. 5,476,786; 5,132,405; and 4,946,778) can be adapted to produce e.g., HMG-CoA reductase-specific single chain antibodies. An additional embodiment of the invention utilizes the techniques described for the construction of Fab expression libraries (Huse et al. (1989) Science 246:1275-1281) to allow rapid and easy identification of monoclonal Fab fragments with the desired specificity for an HMG-CoA reductase, for example, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

Administration of the Therapeutic Compositions of the Present Invention

According to the present invention, the component or components of a therapeutic composition of the invention (including antibodies or fragments thereof) may be introduced topically, parenterally, transmucosally, e.g., orally, nasally, or rectally, or transdermally. When the administration is parenteral, it may be via intravenous injection, and also including, but is not limited to, intra-arteriole, intramuscular, intradermal, subcutaneous, intraperitoneal, intraventricular, and intracranial administration.

In a particular embodiment, the therapeutic compound can be delivered in a vesicle, in particular a liposome (see Langer (1990) Science 249:1527-1533; Treat et al. (1989) in Liposomes in the Therapy of Infectious Disease and Cancer, Lopez-Berestein and Fidler (eds.), Liss: New York, pp. 353-365).

In yet another embodiment, the therapeutic compound can be delivered in a controlled release system. For example, a small organic molecule such as a statin may be administered using intravenous infusion, an implantable osmotic pump, a transdermal patch, liposomes, or other modes of administration. In one embodiment, a pump may be used [see Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials can be used [see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger et al. (1983) J. Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of a therapeutic target, thus requiring only a fraction of the systemic dose (see, e.g., Goodson (1984) in Medical Applications of Controlled Release, supra, vol. 2, pp. 115-138). Other controlled release systems are discussed in the review by Langer (1990) Science 249:1527-1533).

Thus, a therapeutic composition of the present invention can be delivered by intravenous, intraarterial, intraperitoneal, intramuscular, or subcutaneous routes of administration. Alternatively, the therapeutic composition, properly formulated, can be administered by nasal or oral administration. A constant supply of the therapeutic composition can be ensured by providing a therapeutically effective dose (i.e., a dose effective to induce metabolic changes in a subject) at the necessary intervals, e.g., daily, every 12 hours, etc. These parameters will depend on the severity of the infection being treated, other actions, such as diet modification, that are implemented, the weight, age, and sex of the subject, and other criteria, which can be readily determined according to standard good medical practice by those of skill in the art.

A subject in whom administration of the therapeutic composition is an effective therapeutic regiment for bacterial infection is preferably a human, but can be any primate, other mammals or even avians suffering from a bacterial infection, including domestic animals such as dogs and cats, laboratory animals such as rats, rabbits and mice, livestock, such as cattle (including cows), pigs, horses, and goats, and animals maintained in a zoo such as elephants, lions, tigers, and bears. Thus, as can be readily appreciated by one of ordinary skill in the art, the methods and pharmaceutical compositions of the present invention are particularly suited to administration to a number of animal subjects, but particularly humans.

Recently it has been found that the co-ordinated use of L-camitine or an alkanoyl Lcamitine, in which the linear or branched alkanoyl has 2-6 carbon atoms, or one of their pharmaceutically acceptable salts, in conjunction with a statin affords a protective action against statin-induced side-effects (see U.S. Pat. No. 6,245,800 B1, the contents of which are hereby incorporated by reference in its entirety). The present invention therefore provides embodiments in which a given statin is coordinantly administered to a patient with L-camitine or an alkanoyl Lcamitine, in which the linear or branched alkanoyl has 2-6 carbon atoms, or one of their pharmaceutically acceptable salts.

Specific Embodiments

Salmonella is a facultative intracellular pathogen, and its ability to disseminate and cause systemic infection is based upon its survival inside host macrophages. Intracellular Salmonella infection arises from a dynamic interaction between the mammalian host cell and the pathogenic bacterium. Salmonella infection is initiated by the invasion of a single bacterium that resides in a membrane-bound vacoule. Following invasion, the bacterial vacuole interacts briefly with the host cell endocytic machinery but then diverges to form its own privileged niche resistant to normal host cell killing mechanisms. As the bacterium replicates within its vacuole, it manipulates various functions of the host cell by sending it proteins out of the vacuole and into the surrounding cytoplasm. By twenty hours post infection, the original bacterium has divided into over one hundred bacteria each requiring nutrients and additional membrane for construction of the vacuole. Since bacterial vacuoles depend upon a variety of host functions for invasion, replication and exit, the ways in which the Salmonella vacuole require host-specific components were explored. As sterols are important molecules for membrane structure and cell signaling, the role of sterols in Salmonella 's intracellular survival by selectively blocking the different sources of cellular cholesterol were tested.

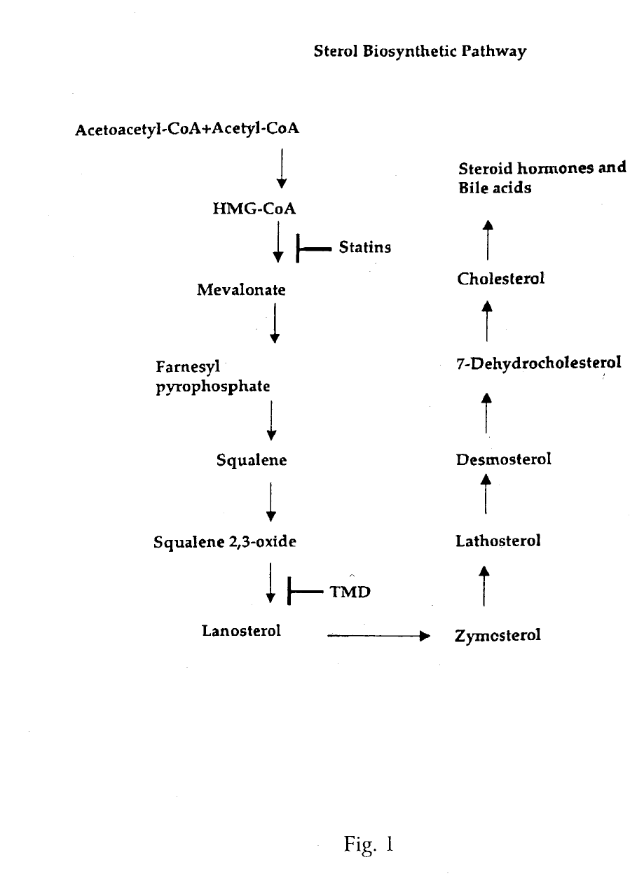

Most of the cholesterol in the blood is carried by low density lipoproteins (LDL). These particles are bound and internalized by specific receptors on the cell surface. Once internalized, LDL is degraded in the lysosomes releasing cholesterol into the cell. Cells also derive cholesterol through de novo biosynthesis. Cell cholesterol levels are exquisitely regulated by the balance of these two sources. Thus, cells deprived of LDL up-regulate biosynthesis and conversely, the inhibition of cholesterol biosynthesis leads to an increase in the number of LDL receptors. HMG CoA reductase is a major enzyme in the cholesterol biosynthetic pathway that has been an important target for a class of drugs called the stains (FIG. 1). Statins inhibit HMG-CoA reductase thereby promoting cellular uptake of LDL from the blood and reducing blood cholesterol levels. These drugs have been widely and successfully used to treat cardiovascular diseases in humans.

There is also evidence that statins could be used to prevent Alzheimer's disease and perhaps kill cancer cells. Extensive research and development efforts have yielded a wide variety of statin compounds, extensive clinical trial data and detailed information about the pharmacokinetic and pharmacodynamic properties of statins. Their effectiveness in blocking sterol biosynthesis, combined with a safe and favorable clinical record, make statins attractive candidates for use in the heretofore unexpected role of treating Salmonella-related disease.

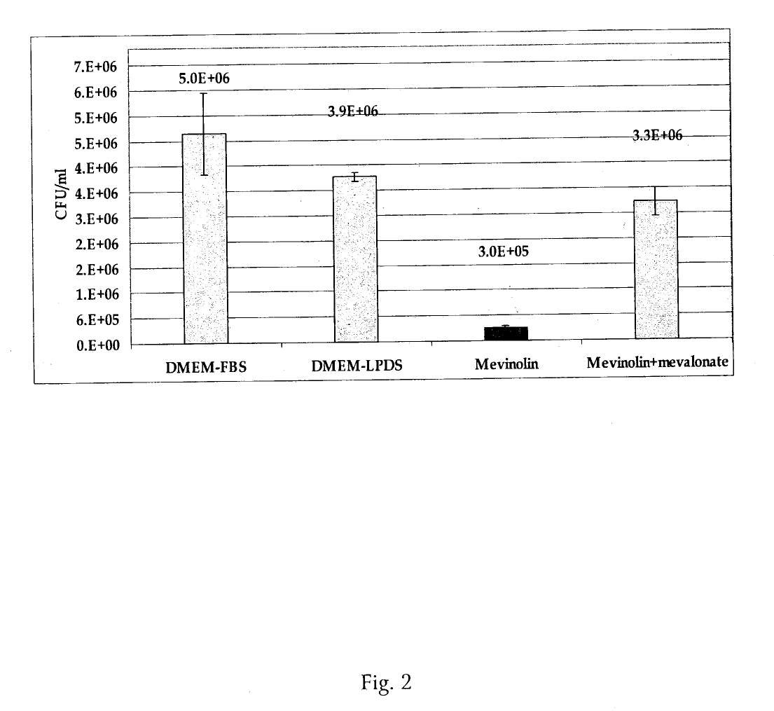

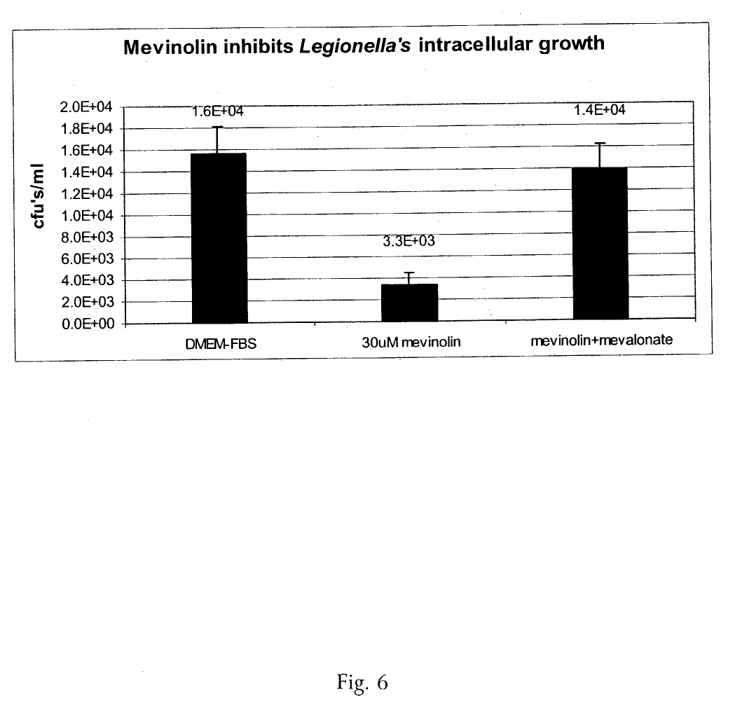

Example 1 below describes the effect of mevinmolin on the intracellular growth of Salmonella. The results (FIG. 2) show that incubating macrophage with a statin to block the sterol biosynthetic pathway of the host cell decreased Salmonella's intracellular survival by a factor of ten. The defect in intracellular growth caused by the statin can be reversed by the addition of mevalonate an intermediate in the sterol biosynthetic pathway (see FIG. 1 and FIG. 2). Similarly, mevinolin has been shown to inhibit intracellular growth of Legionella in human macrophages, whereas this defect in intracellular growth caused by mevinolin also could be reversed by the addition of mevalonate (see FIG. 6).

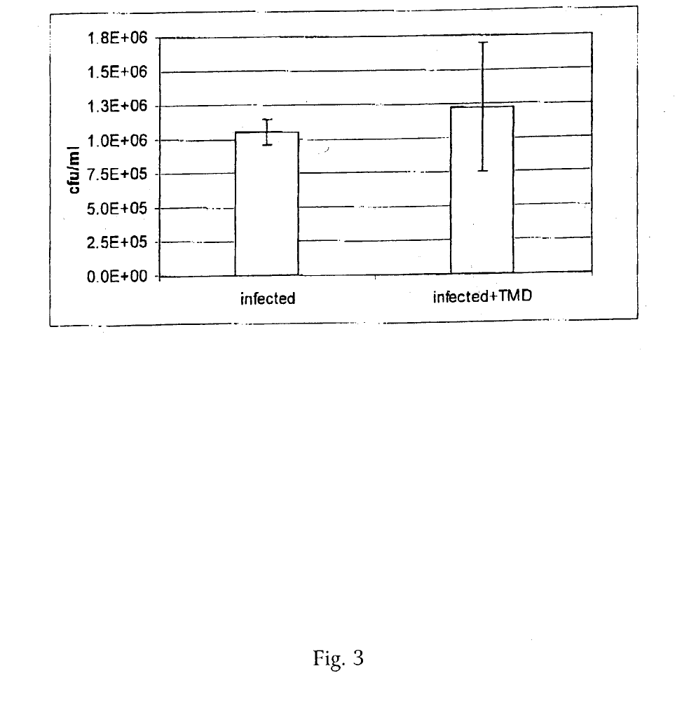

An inhibitor of the conversion of squalene oxide to lanosterol 4,4,10-β trimethyl-trans-decal-3β-ol (TMD), was used to further examine the dependence of bacterial growth on the host cell cholesterol biosynthesis (Example 2). Surprisingly, the inhibition of this early step in the biosynthetic pathway did not lead to a decrease in intracellular survival of Salmonella (FIG. 3). This finding indicates that newly synthesized host cholesterol itself is not required for bacterial growth but rather an early component of the biosynthetic pathway is the crucial factor. This precursor lies between HMG-CoA and squalene 2,3-oxide (see FIG. 1). The isoprenoid pathway which uses mevalonate is an example of one candidate.

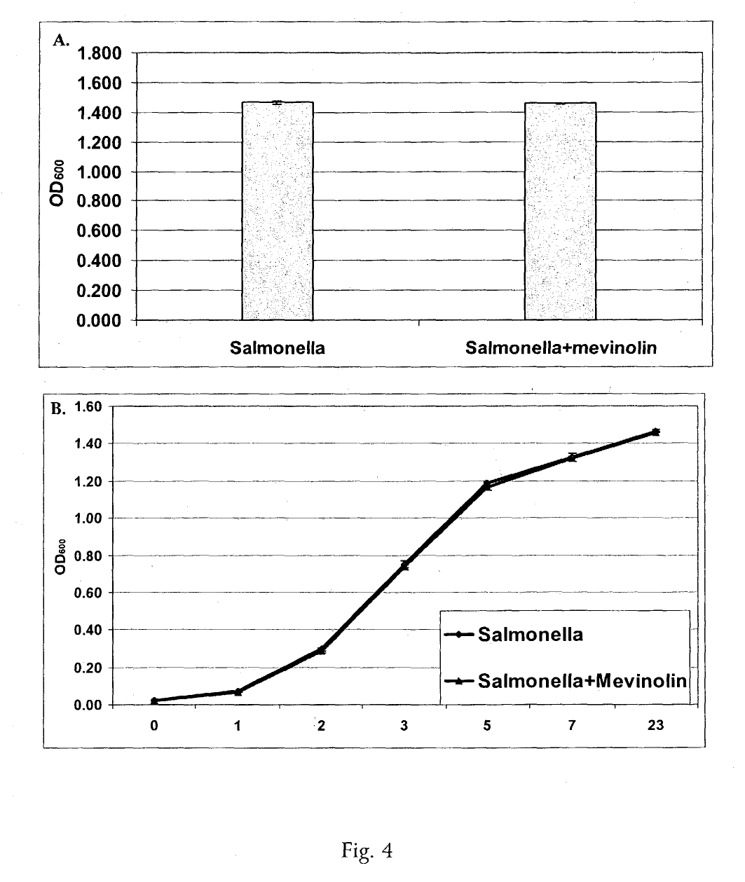

Example 3 describes experiments showing the effect of mevinolin on the extracellular growth of Samonella. The results shown in FIGS. 4A-B show that statin does not affect bacterial growth in liquid culture, indicating that the inhibition is only upon intracellular replication. The effect of mevinolin on host cell viability was studied (Example 4) and it was found that host cell viability is not affected by the statin. Mevinolin was also shown to inhibit the intracellular growth of Legionella in human macrophages (Example 5). Further experiments surprisingly showed that very low concentrations of mevinolin were effective in inhibiting the intracellular growth of S. typhimurium (Examples 6 and 7).

### EXAMPLE

The following examples are put forth so as to provide those of ordinary skill in the art with a complete disclosure and description of how to make and use the assay, screening, and therapeutic methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Mevinolin Inhibits Intracellular Growth of Salmonella in Macrophages

RAW 264.7 macrophages were plated in triplicate at a density of 5×10⁵ cells/well, and incubated for 6 hours prior to infection with various treatments (DMEM-FBS, DMEM-LPDS, DMEM-D-FBS+30 uM Mevinolin, or DMEM-FBS+30 uM Mevinolin, or DMEM-FBS+30 uM Mevinolin+150 uM mevalonate). Cells were infected at a multiplicity of infection of 10 bacteria per cell for 15 minutes at 37° C. After infection, monolayers were incubated in media containing 100 ug/ml gentamicin to kill extracellular bacteria and then maintained in media containing 10 ug/ml for the remainder of the experiment. At 20 hours post infection, the cells were washed three times in PBS, lysed in 1% Triton X-100 and then plated to determine colony forming units. The results are shown in FIG. 2.

### Example 2

### Effect of TMD on Intracellular Growth of Salmonella

RAW 264.7 macrophages were plated in triplicate at a density of 4×10⁵ cells/well, and infected with bacteria at a multiplicity of infection of 10 bacteria per cell for 15 minutes at 37° C.

After infection, the 12 ug/ml of 4,4,10-β trimethyl-trans-decal-3β-ol (TMD) inhibitor was added to the media which also contained 100 ug/ml gentamicin. At 2 hours post infection, the gentamicin concentration was lowered to 10 ug/ml and maintained for the remainder of the experiment. At 20 hours post infection, cells were lysed with 1% Triton X-100 and bacteria were plated for colony forming units. Cells treated with TMD accumulated intermediate precursors and made very little cholesterol.

### Example 3

### Mevinolin Does Not Inhibit Extracellular Growth of Salmonella

Triplicate cultures of Salmonella were grown in a shaking incubator at 37° C. in plain LB, or LB containing 30 uM Mevinolin, and optical density was recorded at 23 hours after inoculation (results shown in FIG. 4A). Triplicate cultures of Salmonella were grown in a shaking incubator at 37° C. in plain LB or in LB containing 30 uM Mevinolin and optical density measurements were used to construct a growth curve (results shown in FIG. 4B).

### Example 4

### Mevinolin Does Not Effect Host Cell Viability

RAW 264.7 macrophages were plated at a density of 1×10⁵ cells/well and each sample represents the mean of six wells. Cells were treated with concentrations of mevinolin at 10 nM (1), 100 nM (2), 500 nM (3), 1 uM (4), 10 uM (5), 50 uM (6), 100 uM (7), 50 uM mevinolin+50 uM mevalonate (8), 50 uM mevinolin+150 uM mevalonate (9), 100 uM mevinolin+50 uM mevalonate (10), and 100 uM mevinolin+150 uM mevalonate (11), for 24 hours at 37° C. and then incubated with Alamar Blue for 3 hours. The ability of the cells to reduce the dye is displayed as the percent reduction compared to untreated (mean of 6 sample wells divided by the mean of 6 untreated wells).

### Example 5

### Mevinolin Effect on Intracellular Growth of Legionella in Human Macrophages

U937 macrophages were plated in triplicate at a density of 5×10⁵ cells/well, and incubated for 6 hours prior to infection with the following treatments: RPMI-FBS, RPMI-FBS+30 uM Mevinolin, or RPMI-FBS+30 uM Mevinolin+150 uM mevalonate. Cells were infected at a multiplicity of infection of 1 bacterium per cell for 1 hour at 37° C. After infection, monolayers were incubated in media containing 100 ug/ml gentamicin to kill extracellular bacteria and then maintained in media containing 10 ug/ml for the remainder of the experiment. At 20 hours post infection, the cells were lysed in 1% Triton X-100 and then plated to determine colony forming units. The results (FIG. 6) show that mevinolin inhibited intracellular growth of Legionella in human macrophages.

### Example 6

### Effect of Physiological Concentration of Mevinolin on S. typhimurium Growth

RAW264.7 macrophages were treated with 30 uM mevinolin starting at 4 hours prior to infection. Infections were done with S. typhimurium (SL1344) at a moi of 10 bacteria per cell. At 13 hours post infection, cells were fixed and processed for TUNEL staining. At least 100 infected and 100 uninfected cells were counted in each monolayer and scored for TUNEL staining. Uninfected cells (black) and infected cells (gray) are shown. Averages of duplicate samples are shown.

### Example 7

### Effect of Physiological Concentration of Mevinolin on S. typhimurium Growth

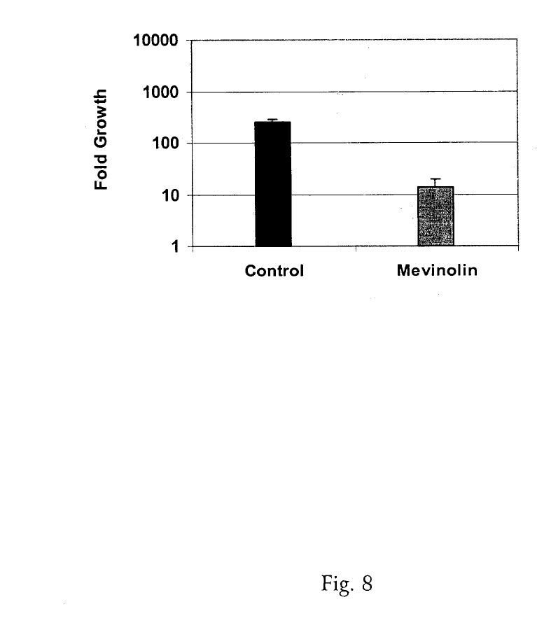

Macrophages were treated with 50 nM mevinolin for 3 days prior to infection. Cells were infected as described above, lysed with Triton X-100 at 2 and 20 hours post infection, and then plated to determine colony forming units. Results are shown in FIG. 8.

What is claimed is:

1. A method of treating a subject having an infection due to an intracellular vacuolar bacterium, comprising administering to the subject an agent capable of inhibiting one or more steps of the sterol biosynthetic pathway between (i) biosynthesis of HMG-CoA from acetoacetyl-CoA and acetyl-CoA, and (ii) conversion of squalene to squalene 2,3-oxide.

2. The method of claim 1, wherein the agent inhibits the conversion of HMG-CoA to mevalonate.

3. The method of claim 1, wherein the agent is an inhibitor of HMG-CoA reductase.

4. The method of claim 1, wherein the intracellular vacuolar bacterium is selected from a genus consisting Salmonella, Legionella, Mycobacterium, Coxiella, Chlamydia, and Campylobacter.

5. The method of claim 4, wherein the bacterium is Salmonella enterica.

6. The method of claim 5, wherein the bacterium is Serovar typhimurium.

7. The method of claim 4, wherein the bacterium is Legionella pneumophila.

8. The method of claim 1, wherein the agent is a small organic compound.

9. The method of claim 8, wherein the small organic compound is a statin.

10. The method of claim 9, wherein the statin is selected from the group consisting of mevinolin, fluvastatin, cerivastatin, atorvastatin, simvastatin and pravastatin.

11. The method of claim 10, wherein the statin is mevinolin.

12. The method of claim 11, further comprising administering mevinolin with L-carnitine or an alkanoyl L-camitine, wherein L-camitine comprises a linear or branched alkanoyl having 2-6 carbon atoms, or a pharmaceutically acceptable salt thereof.

13. The method of claim 1, wherein the subject is a human.

14. The method of claim 1, wherein the agent is a statin administered in a physiological dose of up to 100 nM.

15. The method of claim 14, wherein the dose is 50 nM.

16. A method of ameliorating an infection due to an intracellular vacuolar bacterium, comprising administering to the subject an agent capable of inhibiting one or more steps of the sterol biosynthetic pathway between (i) biosynthesis of HMG-CoA from acetoacetyl-CoA and acetyl-CoA, and (ii) conversion of squalene to squalene 2,3-oxide.

17. A method of treating a subject having an infection due to an intracellular vacuolar bacterium, comprising administering to the subject a therapeutically effective and physiological dose of a statin.

18. The method of claim 17, wherein the statin is selected from the group consisting of mevinolin, fluvastatin, cerivastatin, atorvastatin, simvastatin and pravastatin.

19. The method of claim 17, wherein the intracellular vacuolar bacterium is selected from a genus consisting Salmonella, Legionella, Mycobacterium, Coxiella, Chlamydia, and Campylobacter.

20. The method of claim 16, wherein the dose of statin is up to 100 nM.
